(19) United States
(12) Patent Application Publication
(10) Pub. No.: US 2026/0192100 A1
(43) Pub. Date: Jul. 9, 2026

(54) Tattoo Cartridge and Device

(71) Applicant: Active Needle Technology LTD, Abingdon (GB)

(72) Inventors: Ian Hugh QUIRK, Abingdon (GB); Muhammad Rohaan SADIQ, Abingdon (GB)

(21) Appl. No.: 19/133,892

(22) PCT Filed: Nov. 29, 2023
(86) PCT No.: PCT/GB2023/053091

§ 371 Date: May 29, 2025

(30) Foreign Application Priority Data
Nov. 29, 2022 (GB) 2217906.3

(51) Int. Cl. A61M 37/00

(52) U.S. Cl. CPC A61M 37/0076; A61M 2207/00

(57) ABSTRACT

A tattoo cartridge is disclosed in which the needle assembly can oscillate with respect to a housing assembly at a first frequency, and simultaneously at a second, higher frequency with one or more nodes occurring at one or more nodal points or regions along the needle assembly. The housing assembly is connected to the needle assembly by a connection assembly that is attached to the needle assembly substantially at one or more of the one or more nodal points or regions.

### FIELD OF THE INVENTION

The present invention relates to a tattoo needle cartridge, a tattoo device, and methods of use and manufacture thereof.

### BACKGROUND

A tattoo needle typically comprises a stainless-steel needle array attached (e.g. soldered) to a stainless-steel needle shaft. The needle shaft may, for example, be cylindrical to accommodate a circular needle array pattern, or flat to accommodate a linear needle array pattern.

Tattoo cartridges typically comprise a housing assembly that houses a needle assembly that includes a tattoo needle glued to a PVC needle bar.

Typically, the tattoo needle can project from a distal end of the housing assembly to facilitate tattooing, and the needle bar projects from a proximal end of the housing assembly to couple to a tattoo machine (“tattoo gun”).

Connecting the tattoo cartridge to a tattoo machine couples the needle bar to an oscillator of the tattoo machine, such that oscillations generated by the tattoo machine cause the needle assembly to oscillate longitudinally (along its (longitudinal) axis) with respect to the housing at low frequencies (about 50 to 150 Hz), with amplitudes of a few millimetres, which can cause the needle array to penetrate skin to the level of the dermis, where ink can be retained, e.g. permanently.

The inventors believe that there remains scope for improvements to tattoo cartridges and tattoo devices.

### SUMMARY

According to an aspect, there is provided a tattoo device comprising:
a tattoo cartridge comprising a needle assembly and a housing assembly; and
a tattoo machine configured to oscillate the needle assembly relative to the housing assembly at a first frequency, and to simultaneously vibrate the needle assembly at a second, higher frequency;
wherein the needle assembly is connected to the housing assembly by a connection assembly such that the needle assembly can oscillate with respect to the housing assembly at the first frequency; and
wherein the needle assembly is configured such that in response to being vibrated at the second, higher frequency (by the tattoo machine), an acoustic standing wave can occur along the needle assembly, and wherein the connection assembly is attached to the needle assembly substantially at one or more nodal points or regions of the acoustic standing wave.

According to an aspect, there is provided a tattoo cartridge for use with a tattoo machine that comprises one or more oscillators configured to oscillate a needle assembly of the tattoo cartridge at a first frequency, and to simultaneously vibrate the needle assembly at a second, higher frequency (when the tattoo cartridge is connected to the tattoo machine); the tattoo cartridge comprising:
a housing assembly; and
a needle assembly connected to the housing assembly by a connection assembly such that the needle assembly can oscillate with respect to the housing assembly at the first frequency;
wherein the needle assembly is configured such that in response to being vibrated at the second frequency, an acoustic standing wave can occur along the needle assembly, and wherein the connection assembly is attached to the needle assembly substantially at one or more nodal points or regions of the acoustic standing wave.

According to an aspect, there is provided a tattoo machine comprising:
a cartridge connector configured to receive a tattoo cartridge comprising a needle assembly and a housing assembly; and
one or more oscillators configured to oscillate a needle assembly of a tattoo cartridge connected to the cartridge connector at a first frequency, and to simultaneously vibrate the needle assembly at a second, higher frequency.

According to an aspect, there is provided a tattoo cartridge comprising:
a housing assembly; and
a needle assembly connected to the housing assembly by a connection assembly such that the needle assembly can oscillate with respect to the housing assembly at a first frequency;
wherein the needle assembly is configured to oscillate at a second, higher frequency with one or more nodes occurring at one or more nodal points or regions along the needle assembly, and the connection assembly is attached to the needle assembly substantially at one or more of the one or more nodal points or regions.

Embodiments of the present invention relate to a tattoo cartridge for applying tattoos. In embodiments, the tattoo cartridge includes a needle assembly that extends along a longitudinal axis between a first (proximal) end and a second (distal) end. In embodiments, the first end is configured to couple to a tattoo machine, and the second end is a sharps end for tattooing. In embodiments, the tattoo cartridge includes a housing assembly that houses the needle assembly.

In embodiments, the housing assembly is connected to the needle assembly by a connection assembly such that the needle assembly can oscillate (reciprocate) longitudinally (along its longitudinal axis) with respect to the housing assembly at a first, relatively low frequency (e.g. 1-1000 Hz, e.g. 50-150 Hz), e.g. such that the sharps end of the needle assembly (projecting from a distal end of the housing assembly) can penetrate skin to apply a tattoo. The tattoo cartridge is, in embodiments, removably connectable to a (the) tattoo machine (at its proximal end), e.g. such that when the tattoo cartridge is connected to the tattoo machine, the first (proximal) end of the needle assembly couples to the tattoo machine, such that the tattoo machine can induce the low frequency longitudinal oscillations (reciprocal movement) in the needle assembly.

The tattoo cartridge of embodiments of the present invention is furthermore optimised to efficiently transmit higher frequency (e.g. ≥5 kHz, e.g. ultrasonic) longitudinal oscillations (acoustic waves) along the needle assembly. This is facilitated, in embodiments of the present invention, by the needle assembly being configured such that second, higher frequency (e.g. ≥5 kHz, e.g. ultrasonic) longitudinal oscillations (vibrations) can be simultaneously (i.e. simultaneously with the first, lower frequency) induced (by the tattoo machine) in the needle assembly, with a node (i.e. amplitude minimum) of the higher frequency oscillations (acoustic standing wave) occurring at a nodal point or region along the needle assembly axis (or with plural nodes occurring at plural nodal points or regions along the needle assembly axis). Thus, the needle assembly of embodiments of the present invention is configured such that a high frequency (e.g. ≥5 kHz, e.g. ultrasonic) acoustic standing wave can be induced in the needle assembly. (Only) the connection assembly is then attached to the needle assembly (only) at a nodal point(s) or region(s) of the second, higher frequency oscillations (acoustic standing wave). Thus, the only element(s) fixed to the needle assembly such that the element(s) cannot move longitudinally with respect to the needle assembly may be fixed to the needle assembly at a (respective) nodal point or region.

The Applicant has found that inducing high frequency (e.g. ≥5 kHz, e.g. ultrasonic) longitudinal oscillations (vibrations) in a tattoo needle (simultaneously with low frequency (e.g. ≤1 kHz) longitudinal oscillations) can reduce the insertion force required to penetrate skin layers, and thus reduce trauma and pain sensations that can occur when applying a tattoo. The inventors have found, however, that conventional tattoo cartridge designs tend to strongly dampen such high frequency oscillations (vibrations), such that driving one end of the needle assembly at high frequencies tends not to result in high frequency oscillations (vibrations) sufficient to reduce trauma or pain sensations occurring at the other, sharps end of the needle assembly.

The inventors have found that by limiting (longitudinally fixed) attachment points to the needle assembly to nodes of high frequency (e.g. ≥5 kHz, e.g. ultrasonic) longitudinal oscillations (acoustic standing waves), dampening of the high frequency oscillations resulting e.g. from interaction between the housing assembly and needle assembly, can be minimised, and thus transmission of high frequency oscillations (acoustic waves) along the needle assembly can be optimised. As will be discussed in more detail below, this can then facilitate the trauma and pain reduction benefits associated with high frequency oscillations (vibrations) in the context of a cartridge-based tattoo device.

It will be appreciated, therefore, that the present invention provides an improved tattoo cartridge and tattoo device.

There may be only a single node (of the acoustic standing wave) occurring on the needle assembly, or plural (e.g. two) nodes may occur at different longitudinal positions (nodal points) along the needle axis. For example, the needle assembly may have a half-wavelength design with only one node, or a full-wavelength design with two nodes. In embodiments, the needle assembly is configured such that anti-nodes (i.e. amplitude maxima) occur at the first (proximal) end and the second (distal), sharps end of the needle assembly, and (the) one or more nodes occur between the anti-nodes at the first (proximal) end and the second (distal), sharps end. Correspondingly, the connection assembly may be attached to the needle assembly at only one nodal point or region, or at plural different nodal points or regions.

The connection assembly may be or comprise an elastic member.

The tattoo cartridge may be configured such that the needle assembly can move longitudinally relative to the housing assembly between at least a first (retracted) position in which the housing assembly covers the sharps end of the needle assembly, and a second (extended) position in which the sharps end of the needle assembly is exposed (projects from a distal end of the housing assembly). The housing assembly may be connected to the needle assembly via the elastic member such that when the needle assembly moves from the first position to or towards the second position, the elastic member provides a restoring force that acts to restore the position of the needle assembly to or towards the first position.

The tattoo cartridge may be configured such that when the tattoo cartridge is not connected to a tattoo machine, the needle assembly is biased to the first (retracted) position. The tattoo cartridge may be configured such that when the tattoo cartridge is connected to a tattoo machine, the needle assembly moves from the first (retracted) position to an intermediate position (in between the first and second positions). Connecting the tattoo cartridge to a tattoo machine may thus pre-strain the elastic member. The pre-straining may be such that the elastic member provides a greater restoring force than without pre-straining. This can improve mechanical and/or (ultrasonic) acoustic coupling to a (the) high frequency (e.g. ultrasound) oscillator of a (the) tattoo machine. The pre-straining distance (the distance between the first and intermediate positions) may be 1-6 mm, such as about 2 mm. The pre-straining distance may be user adjustable.

The elastic member may comprise a spring. In embodiments, the elastic member is impermeable. The elastic member may be an impermeable membrane, e.g. made of rubber, plastic, silicone. The elastic member (e.g. membrane) may be a substantially frustoconical member. In embodiments, the elastic member is a silicone membrane having walls with a thickness of at least 0.3 mm, such as at least 0.4 mm, such as at least 0.5 mm. The elastic member (e.g. membrane) may optionally comprise one or more stiffening features, e.g. in the form of one or more ribs or gussets.

The elastic member may be attached (directly) to the needle assembly (substantially) at a nodal point or region (of (the) one or more nodal points or regions). The needle assembly may comprise a surface, e.g. flange or shoulder, configured to hold (one end of) the elastic member at a nodal point or region.

Alternatively, the connection assembly may comprise a first member positioned (substantially) at a nodal point or region (of (the) one or more nodal points or regions), and the elastic member may be attached (e.g. directly) to the first member. The first member may be formed integrally with the needle assembly, or formed separately and attached (longitudinally fixed) to the needle assembly at the nodal point or region. The first member may be a substantially tubular member, e.g. grommet or collar, e.g. surrounding (and attached to) the needle assembly (at the nodal point or region). The first member may be made of metal, plastic, or another suitable material. The first member may comprise a surface, e.g. flange or shoulder, configured to hold (one end of) the elastic member.

The housing assembly may comprise a substantially tubular shell and a cap. The shell may (coaxially) surround the needle assembly. The shell may be closed at an (proximal) end by the cap. The elastic member may be attached to the cap.

A first (e.g. proximal) end of the elastic member may be attached (longitudinally fixed) to the (cap of the) housing assembly and a second (e.g. distal) end of the elastic member may be attached (longitudinally fixed) to the first member or the needle assembly.

The housing assembly may comprise a tip. The shell may be closed at an (distal) end by the tip. The tip may be selected from a plurality of different tip types, wherein each tip type is configured to accommodate a different type of tattoo needle.

The first member may be sized (radially) so as to engage an inner surface of the (shell of the) housing assembly so that the needle assembly is held coaxially with the (shell of the) housing assembly.

The tattoo cartridge may comprise one or more second members sized (radially) so as to engage an inner surface of the (shell of the) housing assembly so that the needle assembly is held coaxially with the (shell of the) housing assembly. A second member may be a tubular bush surrounding the needle assembly and/or first member.

The needle assembly may comprise (be formed from) metal along (substantially) its entire length (from the first (proximal) end to the second (distal), sharps end).

According to an aspect, there is provided a tattoo cartridge comprising:
a needle assembly extending from a first end configured to couple to a tattoo machine, to a second, sharps end;
wherein the needle assembly comprises metal along substantially its entire length.

As discussed above, the tattoo cartridge of embodiments of the present invention is optimised to efficiently transmit high frequency (e.g. ≥5 kHz, e.g. ultrasonic) longitudinal oscillations (acoustic waves) along the needle assembly. This is facilitated, in embodiments of the present invention, by the needle assembly comprising metal (e.g. stainless-steel) along substantially its entire length, e.g. as opposed to conventional tattoo cartridge designs in which typically the needle assembly has a stainless-steel tattoo needle and a PVC needle bar. In embodiments, the arrangement is such that high frequency (e.g. ≥5 kHz, e.g. ultrasonic) longitudinal oscillations (acoustic waves) can propagate along most or all of the longitudinal length of the needle assembly (from the first end to the second, sharps end) through metal.

The inventors have found that forming (substantially) the entire length of the needle assembly from a metal, such as stainless-steel, can allow for significantly improved transmission of high frequency longitudinal oscillations (acoustic waves) along the needle assembly, and improved acoustic coupling of the needle assembly to a high frequency (e.g. ultrasound) oscillator of a tattoo machine. As will be discussed in more detail below, this can then facilitate the trauma and pain reduction benefits associated with high frequency oscillations (vibrations) in the context of a cartridge-based tattoo device.

These aspects and embodiments can, and in embodiments do, include one or more, and in an embodiment all, of the features of other aspects and embodiments described herein, as appropriate. For example, the tattoo cartridge may comprise a housing assembly connected to the metal needle assembly (only) at a nodal point(s) or region(s) (of an acoustic standing wave).

The needle assembly metal can be any suitable metal (including elements, alloys, compounds, etc.). The same (e.g. type of and/or composition of) metal may be used along the entire needle assembly length, or different (e.g. types of and/or compositions of) metal may be used. In embodiments, the metal is stainless-steel, titanium, nickel titanium (nitinol), or aluminium.

The needle assembly may comprise a tattoo needle and a needle bar. The tattoo needle and the needle bar may be made (entirely) of metal. The tattoo needle and the needle bar may be made from the same or different (e.g. types of and/or compositions of) metal. In embodiments, the tattoo needle is made from stainless-steel, titanium, nickel titanium (nitinol) or aluminium, and the needle bar is made from stainless-steel, titanium, nickel titanium (nitinol) or aluminium.

The tattoo needle may extend along the longitudinal axis between a first end and the second (distal), sharps end of the needle assembly. The tattoo needle may comprise a (metal) needle array attached to a (metal) shaft (at the second (distal), sharps end). The needle array may comprise one or more needle points, e.g. arranged in a round or flat configuration. Correspondingly, the tattoo needle shaft may be substantially cylindrical or flat. The sharps end of the (tattoo needle of the) needle assembly may be able to project from the distal end of the housing assembly for tattooing.

The needle bar may extend along the longitudinal axis between the first (proximal) end of the needle assembly and a second end. The first (metal) end of the (needle bar of the) needle assembly may be configured to couple (directly) to an interface of the tattoo machine. The first (metal) end of the (needle bar of the) needle assembly may project from the proximal end of the housing assembly for coupling to the tattoo machine (“tattoo gun”). The needle bar may be substantially cylindrical.

The tattoo needle and the needle bar may be formed integrally.

Alternatively, the tattoo needle and the needle bar may be formed separately and attached to each other. The second end of the needle bar may be attached to the first end of the tattoo needle. The tattoo needle and the needle bar may be attached to each other by any suitable method, such as by using a fixing (e.g. a collet), welding (e.g. laser welding), adhesive (e.g. epoxy), or any combination thereof.

One of the first end of the tattoo needle and the second end of the needle bar may comprise a cavity, and the other of the first end of the tattoo needle and the second end of the needle bar may be received (and attached) within the cavity.

The cavity may be formed in the needle bar and be configured to receive different types of tattoo needle shaft. For example, the cavity may be shaped to receive both cylindrical and flat tattoo needle shafts.

The (e.g. metal) needle assembly may comprise a nodal region that has a greater mass per unit (longitudinal) length than other regions of the needle assembly, e.g. regions on either side of the nodal region, e.g. the needle and/or a shaft region of the needle bar. The nodal region may have a greater diameter than the other regions. The needle assembly may be configured such that a node (of (the) one or more nodes) occurs within the (heavier) nodal region. The shaft region may be at the first end of the needle bar, and the nodal region may be at the second end of the needle bar. The cavity may be formed in the nodal region. The connection assembly may be attached to the needle assembly at the nodal region. For example, the elastic member may be attached (directly) to the nodal region. The (needle bar of the) needle assembly may comprise a surface, e.g. flange or shoulder, configured to hold (one end of) the elastic member at the nodal region.

The length of the needle assembly (from the first end to the second end) may be 10-100 mm, such as about 50 mm.

The tattoo cartridge may be removably connected/connectable to the tattoo machine. The tattoo machine may comprise a cartridge connector that is configured to (removably) connect to the housing assembly of the tattoo cartridge.

The housing assembly may correspondingly comprise a feature, e.g. flange or shoulder, configured to removably connect to the cartridge connector.

In embodiments, the tattoo machine comprises a first oscillator configured to oscillate (reciprocate) the needle assembly at a (the) first frequency; and a second oscillator configured to simultaneously oscillate (vibrate) the needle assembly at a (the) second, higher frequency.

An aspect provides a tattoo device comprising:
a tattoo cartridge as described above; and
a tattoo machine comprising:
   a first oscillator configured to oscillate (reciprocate) the needle assembly at a (the) first frequency; and
   a second oscillator configured to simultaneously oscillate (vibrate) the needle assembly at a (the) second, higher frequency.

The second oscillator may be configured to oscillate (vibrate) the needle assembly at the second, higher frequency such that (the) one or more nodes occur at (the) one or more nodal points or regions.

An aspect provides a tattoo device comprising:
a tattoo cartridge comprising a needle assembly and a housing assembly; and
a tattoo machine comprising a first oscillator configured to (longitudinally) oscillate the needle assembly (relative to the housing assembly) at a first frequency, and a second oscillator configured to simultaneously (longitudinally) oscillate the needle assembly (relative to the housing assembly) at a second, higher frequency such that one or more nodes occur at one or more nodal points or regions along the needle assembly;
wherein the housing assembly is connected to the needle assembly by a connection assembly that is attached to the needle assembly (substantially) at one or more of the one or more nodal points or regions (of the second, higher frequency oscillations).

The tattoo cartridge may be removably connected/connectable to the tattoo machine.

These aspects and embodiments can, and in embodiments do, include one or more, and in an embodiment all, of the features of other aspects and embodiments described herein, as appropriate.

The low frequency (longitudinal) oscillations (reciprocal movement) (induced in the needle assembly by the first oscillator) may have a frequency of (i.e. the first frequency may be) ≤1 kHz, e.g. 1-1000 Hz, e.g. 5-250 Hz, e.g. 10-150 Hz, e.g. 50-150 Hz. The low frequency (longitudinal) oscillations (induced in the needle assembly by the first oscillator) may have an amplitude (“stroke”) of 0.1-10 mm, e.g. 1-6 mm, e.g. 3-4 mm. The amplitude (“stroke”) may be user adjustable.

The first oscillator may comprise: (i) a coil or pair of coils; (ii) a rotary oscillator; (iii) a pneumatic oscillator; or (iv) a fluid driven oscillator.

The second, high frequency (longitudinal) oscillations (vibrations) (induced in the needle assembly by the second oscillator) may have a frequency of (i.e. the second, higher frequency may be) ≥5 kHz, e.g. 5-200 kHz, e.g. 5-100 kHz, e.g. 10-100 kHz, e.g. 20-100 kHz, e.g. 20-75 kHz, e.g. 25-75 kHz, e.g. about 50 kHz. The second, higher frequency may be an ultrasonic frequency, e.g. ≥20 kHz. The second, higher frequency should be, and in embodiments is, a resonant frequency of the needle assembly. The second, higher frequency should be, and in embodiments is, selected to cause an acoustic standing wave to occur along the needle assembly. The second, higher frequency may be selected to be a resonant frequency of both the needle assembly and the second oscillator. The second, high frequency (longitudinal) oscillations (induced in the needle assembly by the second oscillator) may have a (maximum) amplitude of 0.1-50 μm, and/or ≤2 μm.

The second (high frequency) oscillator (e.g. transducer) may comprise one or more vibratory materials, such as a piezoceramic or a piezocrystal material. The second (high frequency) oscillator may comprise one or more electrodes configured such that application of one or more (AC) voltages (e.g. by a voltage supply of the oscillator) causes the vibratory material(s) to vibrate.

The first and/or second oscillator may be configured to oscillate at a frequency controlled by an operator.

The first and/or second oscillator may be coupled to the (needle bar of the) needle assembly, e.g. by a weld, adhesive, screw fitting, push fit, snap fit, bayonet fitting or another other suitable connection.

The first oscillator may oscillate (reciprocate) the second oscillator (with respect to the housing). Alternatively, the tattoo machine may comprise a flexible coupling, the first and second oscillators may be configured to oscillate the flexible coupling, and the first (proximal) end of the (needle bar of the) needle assembly may be coupled to the flexible coupling (when the tattoo cartridge is connected to the tattoo machine).

The first (metal) end of the (needle bar of the) needle assembly may be rounded. The tattoo machine may comprise a complementary (e.g. metal) socket configured to receive the rounded end and thereby couple the first and/or second oscillator to the needle assembly.

An aspect provides a tattoo device comprising:
a tattoo cartridge comprising a needle assembly extending from a first end to a second, sharps end; and
a tattoo machine comprising at least one oscillator configured to oscillate the needle assembly;
wherein the first end of the needle assembly is rounded, and the tattoo machine comprises a complementary socket configured to receive the first, rounded end of the needle assembly to couple the at least one oscillator to the needle assembly.

These aspects and embodiments can, and in embodiments do, include one or more, and in an embodiment all, of the features of other aspects and embodiments described herein, as appropriate.

Another aspect provides a method of operating a tattoo device as described above, the method comprising:
connecting the tattoo cartridge to the tattoo machine.

The method may comprise using the tattoo device to apply a tattoo.

Another aspect provides a non-therapeutic method of applying a tattoo, the method comprising applying a tattoo using a tattoo cartridge or tattoo device as described above.

Another aspect provides a method of manufacturing a tattoo cartridge a described above, the method comprising:
attaching the connection assembly to the needle assembly substantially at one or more of the one or more nodal points or regions.

The needle assembly may comprise (be formed from) metal (along (substantially) its entire longitudinal length).

These aspects and embodiments can, and in embodiments do, include one or more, and in an embodiment all, of the features of other aspects and embodiments described herein, as appropriate.

The method may comprise configuring the needle assembly based on numerical analysis (e.g. finite element analysis), and/or electro-mechanical and/or vibration analysis. The method may be iterative. The (e.g. numerical) analysis may comprise an analysis of the system comprising the tattoo cartridge connected to the tattoo machine.

Thus, the location of the one or more nodes may be determined, and the connection assembly may be attached to the needle assembly substantially at one or more of the one or more determined nodal points or regions. Thus, the connection assembly may be attached (longitudinally fixed) to the needle assembly (only) at a location (or locations) at which a node is expected to occur.

The tattoo machine may be holdable, e.g. in the form of a pen device. The pen/machine may comprise a housing that forms a handle/grip.

According to an aspect, there is provided a device comprising:
a cartridge comprising a needle assembly and a housing assembly; and
a pen/machine configured to oscillate the needle assembly relative to the housing assembly at a first frequency, and to simultaneously vibrate the needle assembly at a second, higher frequency (when the cartridge is connected to the pen/machine);
wherein the needle assembly is connected to the housing assembly by a connection assembly such that the needle assembly can oscillate with respect to the housing assembly at the first frequency; and
wherein the needle assembly is configured such that in response to being vibrated at the second, higher frequency (by the pen/machine), an acoustic standing wave can occur along the needle assembly. The connection assembly may be attached to the needle assembly substantially at one or more nodal points or regions of the acoustic standing wave. The needle assembly may comprise (be formed from) metal along (substantially) its entire length.

According to an aspect, there is provided a cartridge for use with a pen/machine that comprises one or more oscillators configured to oscillate a needle assembly of the cartridge at a first frequency, and to simultaneously vibrate the needle assembly at a second, higher frequency (when the cartridge is connected to the pen/machine); the cartridge comprising:
a housing assembly; and
a needle assembly connected to the housing assembly by a connection assembly such that the needle assembly can oscillate with respect to the housing assembly at the first frequency;
wherein the needle assembly is configured such that in response to being vibrated at the second frequency, an acoustic standing wave can occur along the needle assembly. The connection assembly may be attached to the needle assembly substantially at one or more nodal points or regions of the acoustic standing wave. The needle assembly may comprise (be formed from) metal along (substantially) its entire length.

According to an aspect, there is provided a pen/machine comprising:
a cartridge connector configured to receive a cartridge comprising a needle assembly and a housing assembly; and
one or more oscillators configured to oscillate a needle assembly of a cartridge connected to the cartridge connector at a first frequency, and to simultaneously vibrate the needle assembly at a second, higher frequency.

These aspects and embodiments can, and in embodiments do, include one or more, and in an embodiment all, of the features of other aspects and embodiments described herein, as appropriate.

Each aspect described herein can, and in embodiments does, include one or more, and in an embodiment all, of the features of other aspects described herein, as appropriate.

### BRIEF DESCRIPTION OF THE DRAWINGS

Various embodiments of the present invention will now be described by way of example only and with reference to the accompanying drawings, in which:

FIG. 1A and FIG. 1B show tattoo cartridges;

FIG. 2A and FIG. 2B show tattoo needles that may be used in tattoo cartridges according to embodiments;

FIG. 3 shows different tattoo cartridge housing configurations that may be used with different tattoo needles in embodiments;

FIG. 4 shows schematically a metal needle assembly in accordance with an embodiment;

FIG. 5A and FIG. 5B show schematically metal needle assemblies in accordance with embodiments;

FIG. 6A, FIG. 6B, FIG. 6C, FIG. 6D, FIG. 6E and FIG. 6F show metal needle assemblies having a universal connection feature in accordance with embodiments;

FIG. 7A, FIG. 7B, FIG. 7C and FIG. 7D show finite element analyses of metal needle assemblies in accordance with embodiments;

FIG. 8 shows schematically a tattoo cartridge connected to a tattoo machine in accordance with an embodiment;

FIG. 9A, FIG. 9B and FIG. 9C show a tattoo cartridge in accordance with embodiments;

FIG. 10A shows schematically a tattoo cartridge in accordance with an embodiment, and FIG. 10B shows schematically a tattoo cartridge coupled to a high frequency oscillator in accordance with an embodiment;

FIG. 11A, FIG. 11B, FIG. 11C and FIG. 11D show a tattoo cartridge in accordance with embodiments;

FIG. 12A, FIG. 12B, FIG. 12C and FIG. 12D show a metal needle assembly coupled to a high frequency oscillator in accordance with an embodiment;

FIG. 13A shows a conventional tattoo machine-cartridge interface; and FIG. 13B shows a tattoo machine-cartridge interface in accordance with an embodiment;

FIG. 14A and FIG. 14B show a metal needle assembly of a tattoo cartridge coupled to a high frequency oscillator and a low frequency oscillator of a tattoo machine in accordance with an embodiment; and

FIG. 15 shows schematically a design process according to embodiments.

### DETAILED DESCRIPTION

Modern powered tattoo devices typically work by rapidly vibrating a needle in a controlled, longitudinal manner at around 3000 to 9000 times per minute (about 50 Hz to 150 Hz) over a distance of a few millimetres. Such low frequency vibrations allow ink to enter the skin of a subject to the level of the dermis, where it can be retained, e.g. permanently. Typically, a user can control the frequency and depth of penetration to achieve a desired effect.

Techniques for imparting such low frequency vibratory motion to a tattoo needle include the use of a rotary disk driven by a motor, with an off-centre pin, known to as “rotary machine”, and via a spring-based lever/solenoid combination, known as “coil machine”. Less commonly, a tattoo machine may be pneumatically driven, with compressed air driving a cam which rotates. Modern tattoo machines can be wired or wireless (e.g. and referred to as a “tattoo pen”).

It has been recognised that low frequency needle motion of this kind can cause trauma and pain to the subject. Pain is thought to result from the triggering of nerve cells or endings in the dermal layers by the action of the tattoo needle. Pain can be triggered at the site of needle penetration, or nearby e.g. due rippling motion. The triggering of a pain response in this manner is sometimes referred to as “nociception”, as nociceptors are generally responsible for the transmission of such pain signals. Furthermore, trauma and pain may be triggered by friction between tissue and the moving needle. Other types of pain may occur and contribute to a feeling of pain and/or discomfort.

As discussed in their earlier patent application, WO 2020/089658, the entire contents of which is hereby incorporated by reference, the Applicant has found that the degree of trauma and nociception and other pain/discomfort triggers can be reduced by overlaying higher frequency oscillation, in the range 5-200 kHz, onto the lower, base frequency. The high frequency, e.g. ultrasonic, longitudinal vibratory motion has the effect of lowering the insertion forces required to penetrate the skin layers, thereby reducing trauma, nociception and other pain triggers. The high frequency motion can greatly reduce frictional forces between tissue and the needle.

Tattoo devices traditionally use a needle that comprises a long metal rod with a hook to connect to the tattoo machine. However, it is becoming increasingly common to utilise tattoo cartridges, particularly in combination with a rotary machine. Tattoo cartridges can offer a ready to use, simple and effective tattoo needle module.

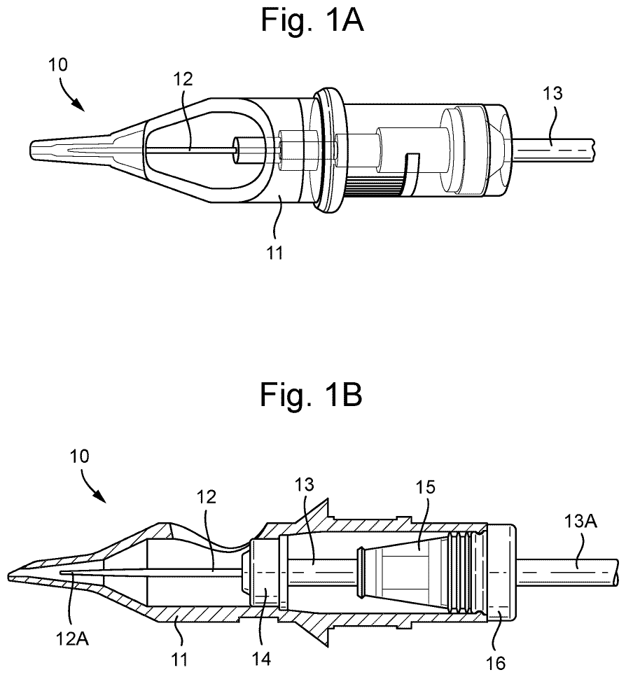

FIGS. 1A and 1B illustrate a tattoo cartridge 10 that includes a shell 11 that houses a needle assembly. FIG. 1A shows a tattoo cartridge 10, and FIG. 1B illustrates a tattoo cartridge 10 with half of the housing 11 removed for the purposes of illustration. The needle assembly includes a stainless-steel tattoo needle 12 glued to a PVC needle bar 13. A limit device 14 is attached to the needle assembly, and is sized to engage an inner surface of the shell 11 to hold the needle assembly coaxially within the shell 11. A flexible elastic membrane 15 is attached at one end to the needle bar 13 and at the other end to a cap 16 that is attached to the shell 11.

FIG. 1 illustrates a relatively simple tattoo needle 12 having a single sharp point at the sharps end 12A of the needle, but it will be appreciated that other types of tattoo needle can be used. For example, the needle 12 may have a needle grouping that includes plural needles grouped together in a round or flat configuration, e.g. to suit a desired tattooing procedure, such as lining, shading, colour packing, etc.

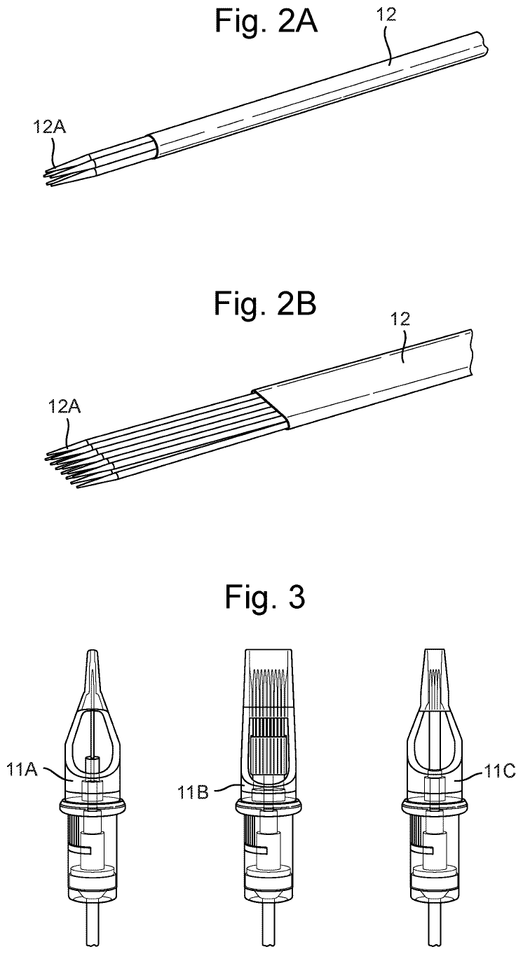

For example, FIG. 2A illustrates a round shader needle 12 having plural needle points grouped in a round configuration at the sharps end 12A, and FIG. 2B illustrates a magnum needle 12 having two rows of needle points at the sharps end 12A. As can be seen in FIG. 2, the round shader has a substantially cylindrical shaft, whereas the shaft of the magnum needle is flat (e.g. closer in shape to a rectangular cuboid with rounded edges). The shell 11 may be configured to fit the needle configuration appropriately. For example, FIG. 3 shows different shell configurations 11A, 11B, 11C that accommodate different types of tattoo needle.

Returning to FIG. 1, the tattoo cartridge 10 is removably connectable to a tattoo machine, and FIG. 1 shows the tattoo cartridge 10 when not connected to a tattoo machine.

As shown in FIG. 1, when the tattoo cartridge 10 is not connected to a tattoo machine (not in use), the needle 12 rests in a retracted position in which the sharps end 12A of the needle 12 is covered by (a distal end of) the shell 11. When the tattoo cartridge 10 is connected to a tattoo machine, a free end 13A of the needle bar 13 (that projects from a proximal end of the shell 11) engages an interface of the tattoo machine which can cause the needle assembly to move longitudinally with respect to the shell 11, to a position in which the sharps end 12A of the needle 12 is exposed (i.e. not covered by the shell 11). This movement causes the flexible membrane 15 to stretch and provide a restoring force which acts to keep the free end 13A of the needle bar 13 engaged with the interface of the tattoo machine, and also to restore the position of the needle 12 to the retracted position when the tattoo cartridge 10 is disconnected from the tattoo machine. This means that the risk of damage to, or accidental injury caused by, the needle 12 when not in use is reduced.

The membrane 15 is impermeable, and provides a fluid-tight barrier between distal and proximal portions of the cartridge. The membrane 15 thus also helps to reduce or eliminate liquid ingress (such as ink or blood) into the tattoo machine.

When the tattoo cartridge 10 is connected to the tattoo machine, the free end 13A of the needle bar 13 is coupled (via the interface) to an oscillator of the tattoo machine, such that low frequency oscillations applied to the free end 13A of the needle bar 13 by the oscillator cause the needle 12 to oscillate (reciprocate) along its longitudinal axis at low frequencies (e.g. 50 Hz to 150 Hz) with amplitudes sufficient for tattooing (e.g. as described above). The connection between tattoo cartridge 10 and tattoo machine is such that the shell 11 is effectively fixed in (longitudinal) position with respect to the tattoo machine, with a periodic force provided by the oscillator and a restoring force provided by the flexible membrane 15 causing the needle assembly to oscillate longitudinally at low frequencies with respect to the shell 11.

The inventors have found, however, that the tattoo cartridge design illustrated by FIG. 1 tends to strongly dampen higher frequency oscillations (vibrations), such that driving the free end 13A of the needle bar 13 at higher, e.g. ultrasonic, frequencies does not result in higher, e.g. ultrasonic, frequency oscillations (vibrations) sufficient to reduce trauma or pain sensations occurring at the sharps end 12A of the needle 12.

Various embodiments accordingly provide an “ultrasound compatible” tattoo cartridge. Embodiments include features described above, and accordingly the following description focuses mainly on differences with respect to arrangements described above. It will be appreciated, however, that features described above can apply to embodiments described below, as appropriate.

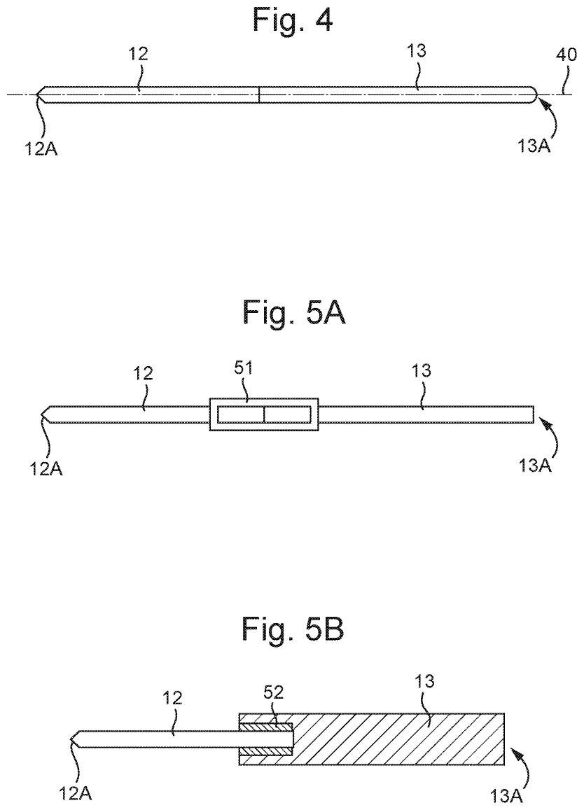

FIG. 4 illustrates a needle assembly that can be used in an “ultrasound compatible” tattoo cartridge according to various embodiments. As shown in FIG. 4, the needle assembly extends along a longitudinal axis 40 between a sharps end 12A of a tattoo needle 12 and a free end 13A of a needle bar 13. FIG. 4 shows the needle assembly (and axis) extending along a substantially straight line, but it will be appreciated that the needle (and axis) may be curved or bent (e.g. in use).

The sharps end 12A of the tattoo needle 12 is configured for tattooing. FIG. 4 illustrates a simple tattoo needle 12 having a single sharp point at the sharps end 12A, but it will be appreciated that other types of tattoo needle, such as those described above, can be used. The free end 13A of the needle bar 13 is configured to couple to (and be driven by) an oscillator(s) of a tattoo machine.

In contrast to the arrangement described above, in the embodiment of FIG. 4, both the needle 12 and the needle bar 13 of the needle assembly are made of stainless-steel. Other metals (including elements, alloys, compounds, etc.) would be possible, such as titanium or nickel titanium (nitinol). Thus, in various embodiments, the tattoo needle and the needle bar are made from metal, e.g. as opposed to the arrangement described above in which the tattoo needle is made from stainless-steel while the needle bar is made from PVC. In various embodiments, the needle assembly comprises metal, such as stainless-steel, along substantially its entire longitudinal length. In embodiments, the arrangement is such that high frequency (e.g. 5-200 kHz) acoustic (e.g. ultrasound) waves can propagate along most or all of the longitudinal length of the needle assembly through the metal.

The inventors have found that the arrangement described above, in which the tattoo needle is made from stainless-steel while the needle bar is made from PVC, tends to strongly dampen transmission of high frequency (e.g. 5-200 kHz) acoustic (e.g. ultrasound) waves, e.g. due to acoustic attenuation properties of PVC, and large acoustic impedance mismatches occurring at the boundary between the needle bar and tattoo needle, and at the interface between the needle bar and oscillator. The inventors have found that forming substantially the entire length of the needle assembly (i.e. at least both the tattoo needle 12 and needle bar 13) from metal, such as stainless-steel, can allow for significantly improved transmission of high frequency acoustic (e.g. ultrasound) waves from the free end of the needle assembly to the other, sharps end, and significantly improved acoustic coupling between the needle assembly and oscillator. In particular, driving the free end 13A of the stainless-steel needle bar 13 at high frequencies (e.g. 5-200 kHz) can result in high frequency (e.g. 5-200 kHz) longitudinal oscillations (vibrations) sufficient to reduce trauma and pain sensations occurring at the sharps end 12A of the stainless-steel tattoo needle 12.

The tattoo needle 12 and needle bar 13 can be formed integrally, or formed separately and attached to each other. The tattoo needle 12 and needle bar 13 can be attached to each other by any suitable method, such as by using a metal fixing (e.g. collet), welding (e.g. laser welding), an adhesive having a relatively high acoustic impedance (e.g. epoxy resin), or any combination thereof. FIG. 5A illustrates an embodiment in which the needle 12 and the needle bar 13 are attached to each other by a metal, e.g. stainless-steel, collet or collar 51. FIG. 5B illustrates an embodiment in which the non-sharps end of the needle 12 is received in a cavity formed in the non-free end of the needle bar 13, and epoxy or a weld 52 in the cavity attaches the needle 12 to the needle bar 13.

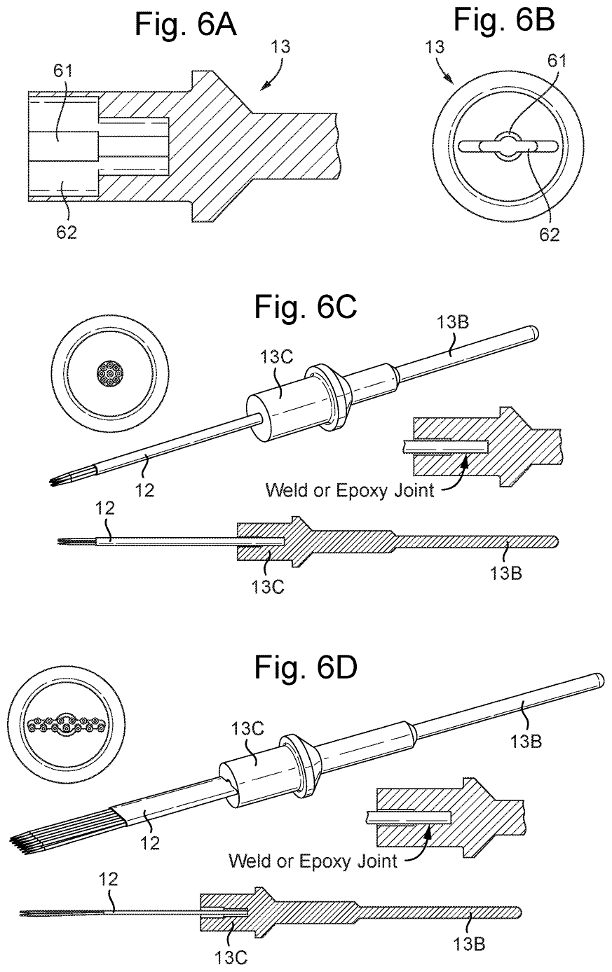

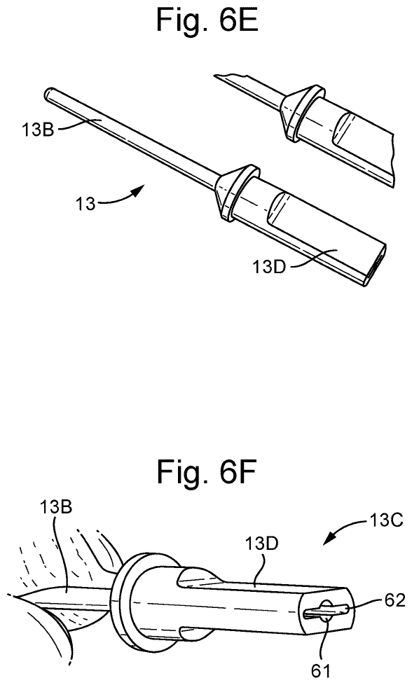

FIG. 6 illustrates embodiments in which the needle bar 13 has a universal cavity design configured to accommodate different types of tattoo needles. As can best be seen in FIGS. 6A, 6B and 6F, in these embodiments, the needle bar cavity includes a first volume 61 shaped to receive a cylindrical tattoo needle shaft, and a second volume 62 shaped to receive a flat needle shaft, e.g. of a magnum needle. In these embodiments, the needle bar 13 includes a shaft section 13B at one end, and a cavity containing section 13C at the other end. To accommodate the cavity, the cavity containing section 13C has a larger diameter than the shaft section 13B. FIG. 6C illustrates the round shader needle 12 of FIG. 2A attached to a needle bar 13 via the universal cavity in the needle bar cavity containing section 13C, and FIG. 6D illustrates the magnum needle 12 of FIG. 2B attached to the needle bar 13 via the universal cavity in the needle bar cavity containing section 13C. In the embodiment illustrated in FIGS. 6A-C, the external shape of the needle bar is substantially radially symmetric along its entire length, but in other embodiments this need not be the case. For example, FIGS. 6E and 6F show an embodiment in which the cavity containing section 13C includes a flat section 13D.

This can reduce the weight of the metal needle bar 13.

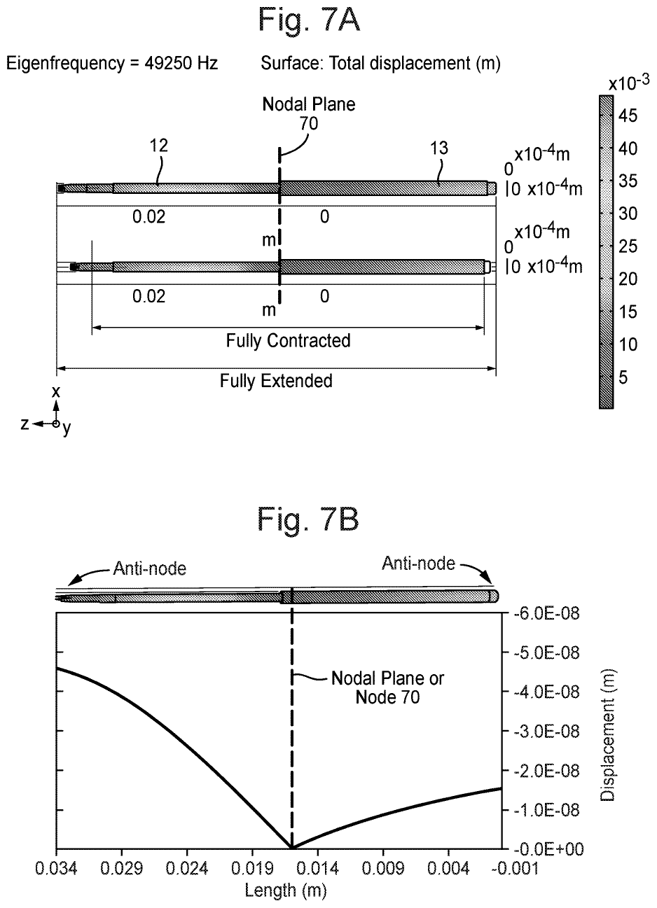

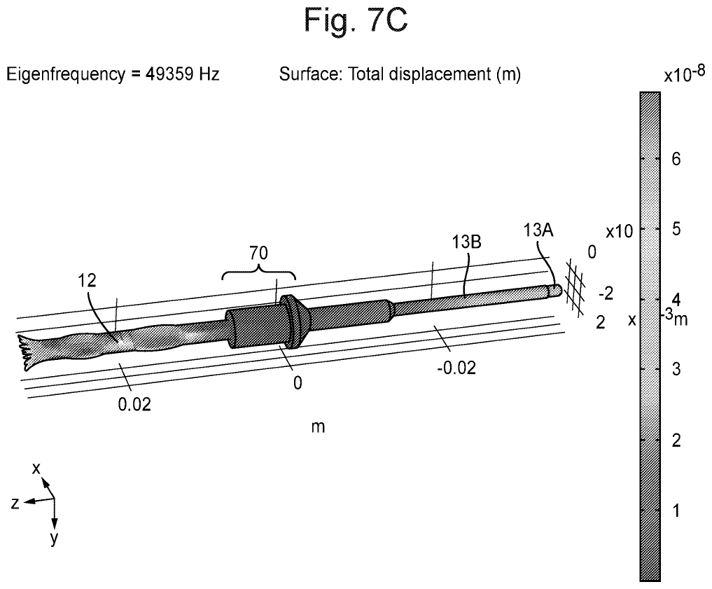

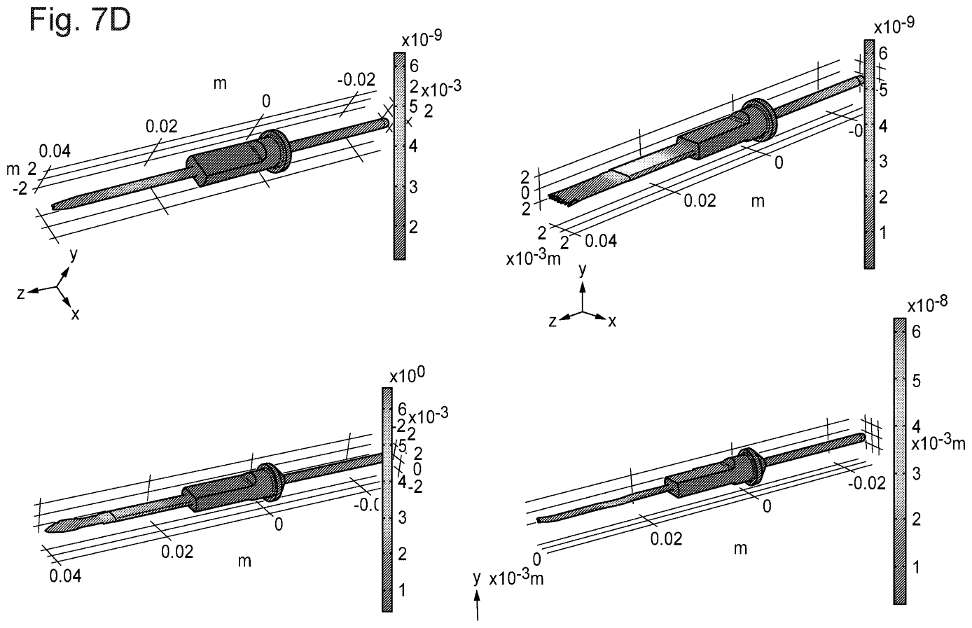

FIGS. 7A and 7B illustrate the results of a finite element analysis in which a stainless-steel needle assembly substantially as shown in FIG. 5B was driven longitudinally at its free end at a resonant frequency of 49.25 kHz. As shown in FIGS. 7A and 7B, in response to driving the needle bar 13 longitudinally at the resonant frequency, an acoustic (i.e. longitudinal) standing wave propagates along the entire longitudinal length of the needle assembly, such that the sharps end of the tattoo needle 12 oscillates longitudinally at the resonant frequency.

As shown in FIGS. 7A and 7B, the resonant high frequency acoustic wave and needle assembly configuration is such that the needle assembly oscillates between maximum longitudinal expansion and maximum longitudinal contraction, with maximum longitudinal amplitudes (i.e. anti-nodes) occurring at the ends of the needle assembly, and a single node 70 (i.e. minimum (zero) longitudinal amplitude) occurring substantially at the middle point of the needle assembly, which in this example corresponds to the longitudinal position (plane) at which the needle 12 and needle bar 13 are attached to each other. However, it will be appreciated that other resonant frequencies and/or needle assembly designs can result in different node and anti-node positions, and e.g. plural nodes occurring.

FIG. 7C illustrates the results of a finite element analysis in which a stainless-steel needle assembly substantially as shown in FIG. 6D was driven longitudinally at its free end 13A at a resonant frequency of 49359 Hz. As shown in FIG. 7C, in this example the needle assembly is configured such that a single node occurs at the cavity containing section 13C of the needle bar. The increased radial diameter of the cavity containing section 13C increases the mass per unit length in this region (as compared to the shaft section 13B and needle 12), which can broaden the amplitude minimum as compared to the embodiment of FIG. 7B, effectively resulting in a nodal region 70 that spans the cavity containing section 13C. This can make the node position less sensitive, e.g. to different needle types being used.

For example, FIG. 7D illustrates the results of finite element analyses of the same universal needle bar 13 connected to four different tattoo needle types. FIG. 7D demonstrates that, as a result of the increased diameter (mass per unit length) in the cavity containing section 13C of the needle bar 13, the position of the nodal region 70 at the cavity containing section 13C of the needle bar 13 is substantially unchanged by different needle types being connected to the universal needle bar 13.

The metal needle assembly is thus, in various embodiments, configured to oscillate (in use) at a high frequency (e.g. of at least 5 kHz) with one or more nodes occurring at one or more nodal points (planes) or regions along the needle assembly. In other words, the needle assembly is configured such that, in response to being oscillated (longitudinally) at a (resonant) high frequency (e.g. of at least 5 kHz) (by an oscillator of a tattoo machine), the needle assembly oscillates (longitudinally) with one or more nodes (longitudinal amplitude minima) occurring at one or more nodal points or regions along the needle assembly. Thus, in various embodiments, the needle assembly is configured such that a high frequency acoustic standing wave can occur along the (entire) length of the needle assembly.

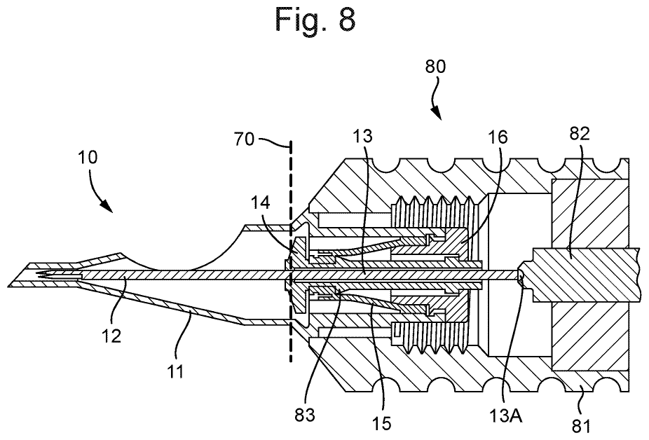

FIG. 8 illustrates an “ultrasound compatible” tattoo cartridge 10 connected to a tattoo machine 80 according to an embodiment. As shown in FIG. 8, the tattoo cartridge 10 includes a shell 11 that houses a needle assembly that includes stainless-steel tattoo needle 12 and stainless-steel needle bar 13. In this embodiment, the tattoo machine 80 comprises a grip 81 housing an ultrasonic transducer 82. FIG. 8 shows the shell 11 removably connected to the tattoo machine housing 81, with the free end 13A of the needle bar 13 coupled to the ultrasonic transducer 82.

The ultrasonic transducer 82 causes the needle assembly to oscillate/vibrate longitudinally at a high (5-200 kHz), resonant frequency, and the tattoo device 80 includes another oscillator (not shown) that simultaneously oscillates/reciprocates the needle assembly longitudinally at a low frequency (<1 kHz). The high frequency is selected to be a resonant frequency of both the ultrasonic transducer 82 and the needle assembly 12, 13, thereby optimising acoustic coupling. Thus, a resonant vibration is superimposed on a low-frequency reciprocal movement.

The transducer 82 may include a metal (e.g. steel) rear mass and a metal (e.g. titanium) front mass, separated by a vibratory material. To generate the high frequency vibrations, any suitable vibratory material may be used, such as a piezoceramic or a piezocrystal material. In response to a stimulus, such as electricity at a defined frequency, the vibratory material of the transducer may vibrate/resonate the front mass, and thus the needle assembly, at the desired frequency.

As in the arrangement of FIG. 1, the tattoo cartridge 10 of FIG. 8 includes a limit device 14, flexible elastic membrane 15 and cap 16. Cap 16 forms part of the housing, and is attached to a proximal end of shell 11. The limit device 14 is attached to the needle assembly, and is sized to engage an inner surface of the shell 11 to hold the needle assembly coaxially within the shell 11.

The flexible membrane 15 provides an impermeable barrier, and a longitudinal restoring force to keep the free end 13A of the needle bar 13 engaged with the ultrasonic transducer 82, and to facilitate the needle assembly oscillating longitudinally with respect to the shell 11 (at low frequencies), e.g. substantially as discussed above.

In contrast to the arrangement of FIG. 1, the flexible membrane 15 is pre-strained so that, when the tattoo cartridge 10 is connected to the tattoo machine 80, the flexible membrane 15 provides a larger force than would otherwise be the case to keep the free end 13A of the needle bar 13 firmly engaged with the ultrasonic transducer 82. This can facilitate good acoustic coupling between the needle bar 13 and transducer (oscillator) 82, thereby facilitating resonant behaviour and improving the transmission of high frequency vibrations from the transducer 82 to the needle assembly.

As shown in FIG. 8, a first (proximal) end of the flexible membrane 15 is connected to cap 16. In contrast to the arrangement of FIG. 1, the second (distal) end of the flexible membrane 15 of the embodiment of FIG. 8 is connected to the limit device 14. In particular, as shown in FIG. 8, the limit device 14 includes a flange 83 that prevents longitudinal movement of the second (distal) end of the flexible membrane 15 with respect to the limit device 14. Thus, the (longitudinal) position of the first end of the flexible membrane 15 is fixed with respect to the housing 11 by being attached to cap 16, and the (longitudinal) position of the second end of the flexible membrane 15 is fixed with respect to the needle assembly by being attached to the limit device 14.

This means that, in contrast to the arrangement shown in FIG. 1 in which the limit device and flexible membrane are connected separately to the needle assembly at different longitudinal positions, the needle assembly of the embodiment of FIG. 8 only has a single (longitudinally fixed) point of attachment to the housing along its longitudinal length.

Furthermore, in the embodiment shown in FIG. 8, the needle assembly is configured substantially as discussed above with reference to FIGS. 7A and 7B, and the limit device 14 is attached to the needle assembly at a longitudinal position on the needle assembly that corresponds to a node 70 of the high frequency oscillations (i.e. a position along the longitudinal axis of the needle assembly at which a longitudinal amplitude of the high frequency (≥5 kHz) longitudinal oscillations (acoustic waves) is (expected to be) at a minimum). Thus, the connection assembly 14, 15 is attached to the needle assembly 12, 13 only at a nodal point corresponding to a high frequency (≥5 kHz) acoustic wave node.

The inventors have found that by limiting points of attachment to the needle assembly to high frequency (≥5 kHz) oscillation nodes, dampening of resonant behaviour and high frequency oscillations resulting e.g. from interaction between the housing and needle assembly can be minimised, and thus transmission of high frequency oscillations along the needle assembly can be improved.

In particular, the inventors have found that forming substantially the entire length of the needle assembly (i.e. at least both the tattoo needle 12 and needle bar 13) from metal, such as stainless-steel (as discussed above), and also attaching the housing to the needle assembly at a nodal point (plane), can provide significantly improved transmission of high frequency (e.g. 5-200 kHz) acoustic (e.g. ultrasound) waves from the free end of the needle assembly to the other, sharps end, e.g. as compared the arrangement shown in FIG. 1. This can then allow the trauma pain reduction benefits associated with high frequency oscillations to be realised in the context of a cartridge-based tattoo device.

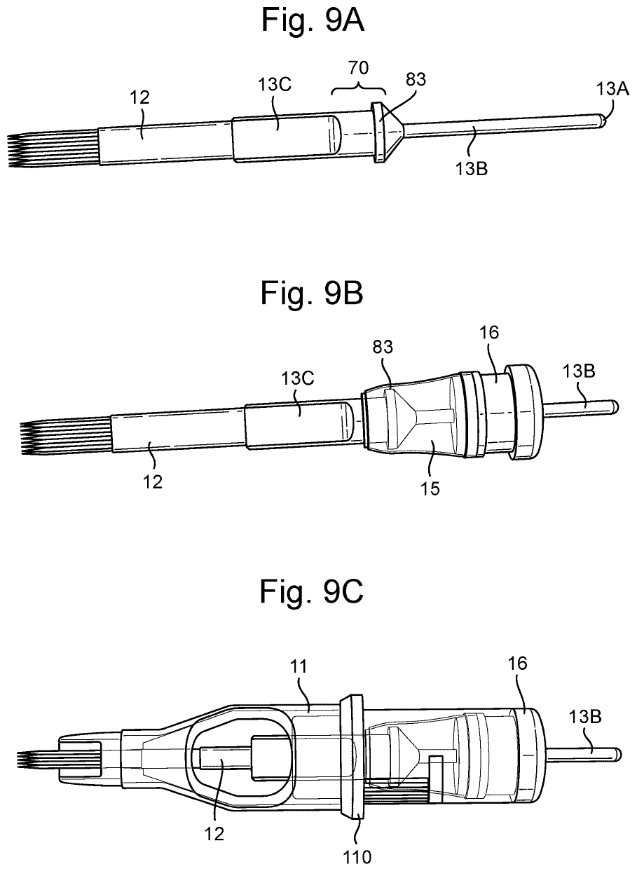

FIG. 9 illustrates another embodiment in which the connection assembly is attached to the needle assembly only at a high frequency nodal region of the needle assembly. As shown in FIG. 9A, in this embodiment the needle assembly comprises a flat tattoo needle 12 that is received within a cavity formed in a cavity containing section 13C of the needle bar.

The needle assembly is configured substantially as discussed above with reference to FIGS. 7C, and thus the needle bar has an increased radial diameter section at which a nodal region 70 occurs. As can be seen in FIGS. 9A and 9B, the needle bar includes an integral flange 83 configured to hold the distal end of the flexible membrane 15 longitudinally at the nodal region 70.

As can be seen in FIGS. 9B and 9C, the cap 16 is then attached to the proximal end of the flexible membrane 15, and the shell 11 is attached to the cap 16. There is thus no direct physical interface between needle assembly 12, 13 and the housing assembly 11, 16. A bushing (not shown) may be fitted between the shell 11 and needle 12, and/or the cap 16 and the needle bar shaft 13B, to help maintain coaxial alignment between the shell 11 and needle 12. The housing may include a surface 110, e.g. flange or shoulder, that interfaces with the tattoo machine.

Thus, in this embodiment, only the distal end of the flexible membrane 15 is fixed to the needle assembly, and the distal end of the flexible membrane 15 is fixed to the needle assembly only at a nodal region.

It will be appreciated that in the embodiment of FIG. 8, the limit device 14 both holds the needle assembly 12, 13 at a nodal point, and is sized to hold the needle assembly coaxially within the shell 11, whereas in the embodiment of FIG. 9 the tattoo cartridge may have a bushing to hold the needle assembly coaxially within the shell 11.

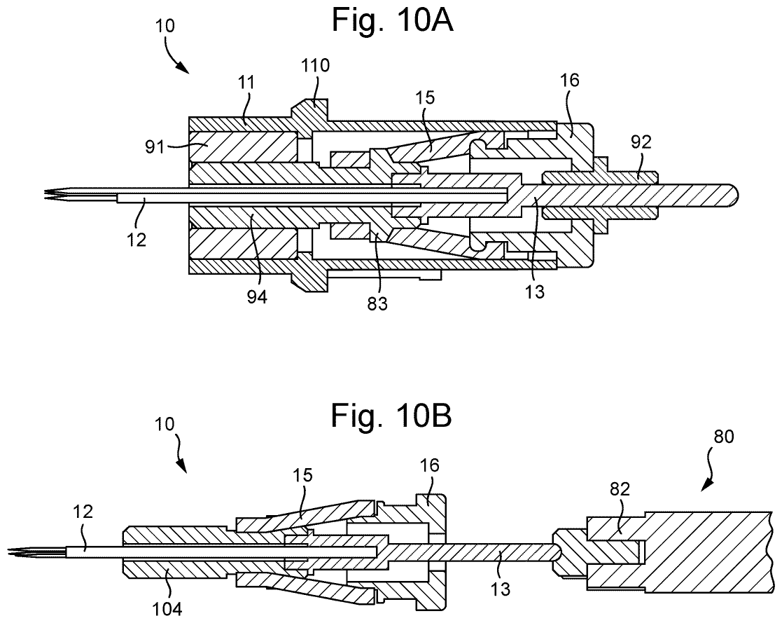

FIG. 10A illustrates an embodiment in which the tattoo cartridge 10 comprises a distal bush 91 and a proximal bush 92 that are sized to hold the needle assembly coaxially within the shell 11.

As shown in FIG. 10A, in this embodiment, cap 16 is attached to shell 11, and the assembly includes a stainless-steel collar 94 positioned at a nodal point. The collar 94 comprises a flange 83 configured to fix one end of the flexible membrane 15, and the other end of the flexible membrane 15 is attached to cap 16. Front bush 91 is a tubular plastic member that is shaped to substantially fill an annular space between collar 94 and shell 11 so as to hold the needle assembly coaxially within the shell 11. Similarly, rear bush 92 is a tubular plastic member that is shaped to substantially fill an annular space between needle bar 13 and cap 16 so as to hold the needle assembly coaxially within the shell 11. Bushes 91, 92 allow the needle assembly 12, 13, 94 to slide past in the longitudinal direction.

Other bush arrangements would be possible.

FIG. 10B illustrates an embodiment similar to that shown in FIG. 10A, but with a plastic collar 104 (tubular member) is attached to the needle assembly 12, 13 at the nodal point. FIG. 10B shows the tattoo needle cartridge 10 connected to a tattoo machine 80 having an ultrasonic transducer 82. Elements of the housing are not shown in FIG. 10B for clarity. In this embodiment, cap 16 is attached to shell 11 (not shown), and plastic collar 104 is attached to the needle assembly 12, 13 substantially at a nodal point or region. Flexible membrane 15 is then attached at one end to the collar 104 at the nodal point and at the other end to cap 16. In other embodiments, a metal grommet may be attached to the needle assembly 12, 13 at a nodal point, and the flexible membrane may be attached to the metal grommet.

FIGS. 9 and 10 illustrate embodiments that have a needle assembly design similar to that shown in FIG. 6. As can be seen in FIGS. 9 and 10, in these embodiments, a (stainless-steel) needle bar 13 has a cavity within which a (stainless-steel) tattoo needle 12 is received and attached. However, it will be appreciated that other needle assembly designs are possible.

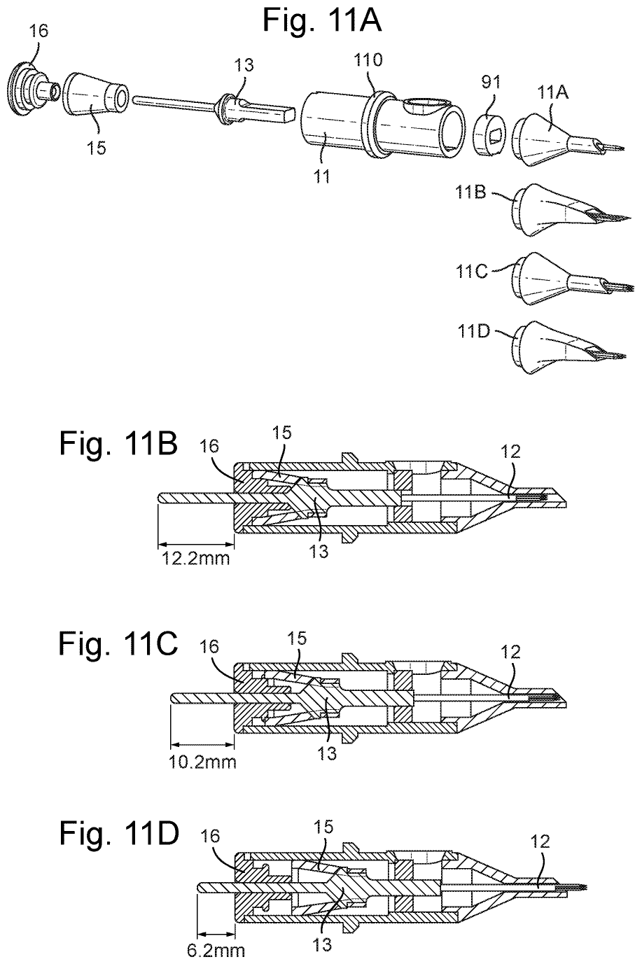

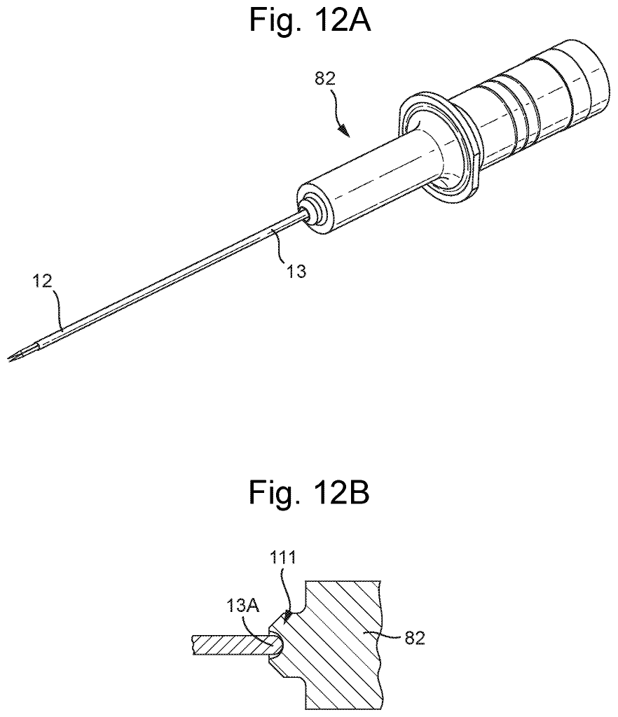

FIG. 11A is an exploded view of a tattoo cartridge according to an embodiment. The tattoo cartridge includes a machined or 3D printed polymer bush 91, and a silicone membrane 15 that attaches to a stainless-steel needle bar 13 and to a 3D printed cap 16. The housing further includes a 3D printed shell 11 and a 3D printed shell tip. As illustrated in FIG. 11A, different shell tips 11A-11D can be attached to the shell 11 to accommodate different needle types.

FIG. 11B shows the tattoo cartridge when not connected to a tattoo machine. As shown in FIG. 11B, in this position, the needle bar 13 rests against the cap 16, which forms a stop, and the sharps end of the needle 12 is covered by the shell tip.

FIG. 11C shows the tattoo cartridge after having been connected to a tattoo machine. As illustrated by FIG. 11C, attaching the tattoo cartridge to the tattoo machine causes the needle assembly to move towards the distal end of the tattoo cartridge, thereby pre-straining the membrane 15 and causing a restoring force that can facilitate good (e.g. ultrasonic) acoustic coupling between the needle assembly and tattoo machine. The inventors have found pre-straining a silicone membrane to 1-6 mm, such as about 2 mm or 3 mm, can provide sufficient force to facilitate good acoustic coupling. The pre-straining distance may be varied depending on membrane thickness, stiffness, etc. In embodiments, the pre-straining distance (the needle “throw”) is user adjustable, e.g. between 1-3 mm. That is, the distance between the needle positions shown in FIGS. 11B and 11C may be adjustable, e.g. such that the level of pre-straining is adjustable.

A restoring force provided by the membrane 15 and low frequency oscillations provided by the tattoo machine can then cause the needle assembly to oscillate (reciprocate) between the positions shown in FIGS. 11C and 11D. As can be seen in FIGS. 11C and 11D, the sharps end of the needle 12 oscillates between retracted and extended positions to facilitate tattooing. In embodiments, the oscillation amplitude (“stroke”) is user adjustable, e.g. between 3-4 mm. That is, the distance between the retracted and extended needle positions shown in FIGS. 11C and 11D may be adjustable.

To guide the determination of pre-strain and stroke values, different commercially available elastic membranes were integrated into a test rig. Starting from 1 mm, strain distance was increased with steps of 1 mm using a position adjustment feature of the rig until a maximum of 7 mm was reached. At each step, an impedance test was carried out to observe changes in frequency, impedance and curve profile. The elastic force provided by the membranes at different strain distances was also measured using a force gauge.

Table 1 illustrates some results of the acoustic impedance measurements for a relatively thin-walled (about 0.3 mm thick), soft silicone membrane (membrane A) and a relatively more thick-walled (about 0.5 mm thick), stiff silicone membrane (membrane B). Table 1 demonstrates that acoustic impedance decreases with increasing pre-strain distance. Furthermore, acoustic impedance is generally lower for a thicker membrane as compared to a thinner membrane (for the same pre-strain distance). Correspondingly, results of the force measurements showed spring force increasing with increasing strain distance, and increasing with membrane wall thickness. The force measurements were compared with theoretically calculated peak spring forces that would be expected to maintain firm metal needle bar-transducer contact throughout a complete oscillation cycle of low frequency (e.g. 150 Hz) reciprocating motion. The measurements and calculations confirmed that selecting a relatively stiffer/thicker membrane can reduce the strain distances required to provide a given force, and thus can allow relatively smaller pre-strain and/or stroke distances to be used.

TABLE 1
results of acoustic impedance measurements for
different membranes and pre-strain distances

| | Strain distance | Frequency | Impedance |
|---|---|---|---|
| Membrane | (mm) | (kHz) | (Ohms) |
| A | 4 | 52.12 | 200 |
| B | 1 | 53.22 | 226 |
| B | 2 | 53.21 | 167 |

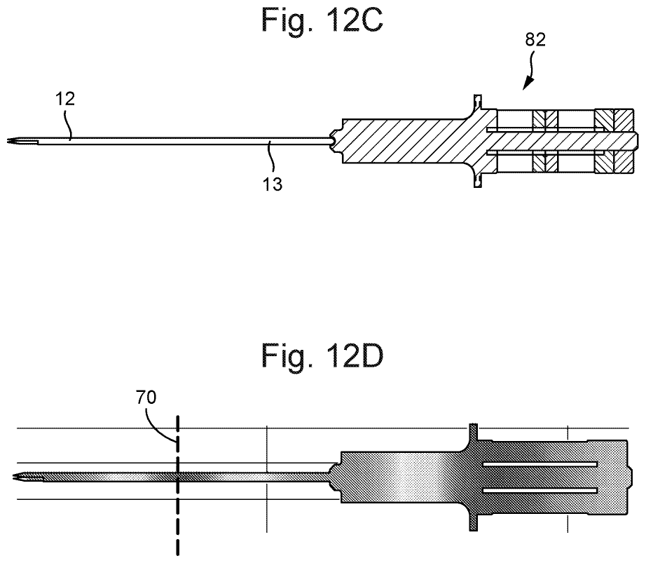

The free end 13A of the needle bar 13 can couple to a high frequency (e.g. ultrasonic) oscillator of a tattoo machine in any suitable manner. FIGS. 12A-D illustrate a stainless-steel needle assembly 12, 13 coupled to an ultrasonic transducer 82 by a pin and socket interface, according to an embodiment. As can best be seen in FIG. 12B, in this embodiment, the metal free end 13A of the needle bar 13 has a rounded, convex, radially symmetric shape, and the ultrasonic transducer 82 includes a metal socket 111 with a complementary, convex, radially symmetric shape. The inventors have found that this arrangement can minimise energy transferred from the transducer 82 into transverse oscillations of the needle assembly 12, 13, and thus maximise energy transferred into desired longitudinal oscillation modes.

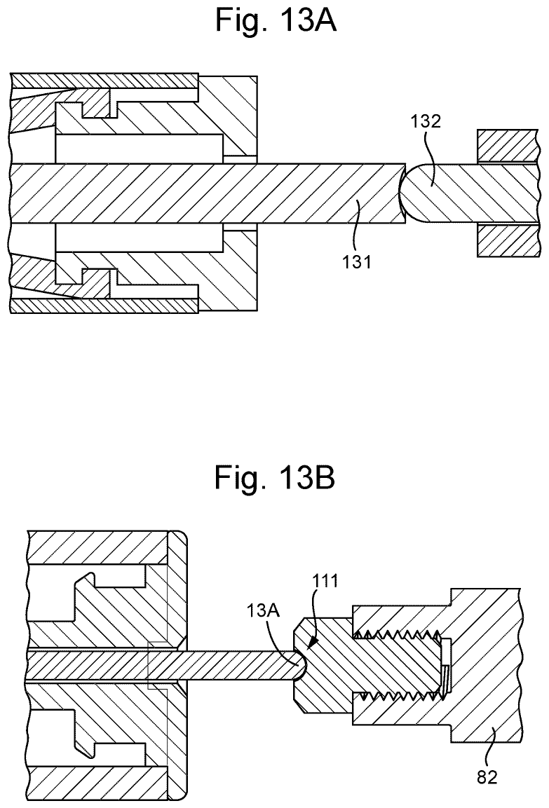

Furthermore, this arrangement increases the area of contact between the metal free end 13A of the needle bar 13 and the socket 111, e.g. as compared to conventional arrangements that have a concave needle bar end. This is demonstrated by FIG. 13. FIG. 13A illustrates a conventional tattoo machine-cartridge interface in which the tattoo machine has a rounded pin 132 that interfaces with a concave dip in the end of the plastic needle bar 131. FIG. 13B illustrates an embodiment in which the tattoo machine has a concave socket 111 that interfaces with the rounded end 13A of a metal needle bar 13. The area of contact between socket 111 and rounded needle bar end 13A has been found to be about 50% larger than the area of contact between pin 132 and needle bar 131 in conventional arrangements. This can improve mechanical and acoustic coupling between needle and transducer.

FIG. 12D illustrates the results of a finite element analysis, showing a node of longitudinal oscillations occurring at a nodal point 70 substantially at the middle point of the needle assembly 12, 13, and anti-nodes occurring at the ends of the needle assembly 12, 13. Other interface arrangements, such as a bayonet interface would be possible. In embodiments, the interface has a quick connect and release function.

In these embodiments, the tattoo machine further includes a low frequency oscillator (e.g. a rotary disk driven by a motor, with an off-centre pin) (not shown), that induces low frequency (<1 kHz) longitudinal oscillations (reciprocal motion) in both the needle assembly 12, 13 and the ultrasonic transducer 82 (relative to the housing). For example, returning to FIG. 8, in this embodiment the ultrasonic transducer 82 oscillates longitudinally at low frequencies with respect to the grip 81.

However, in other embodiments, the ultrasonic transducer 82 may remain substantially stationary with respect to the housing.

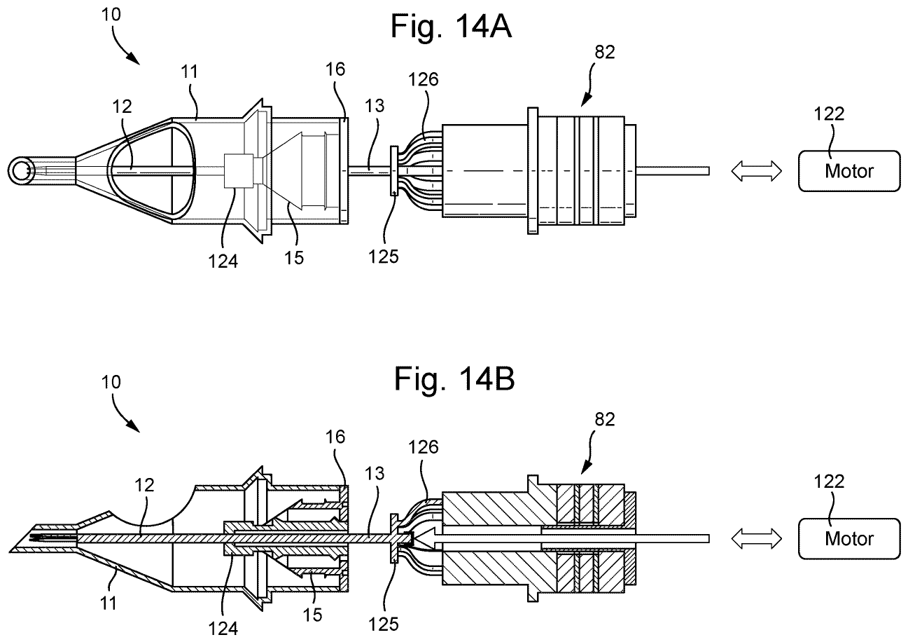

For example, FIGS. 14A and 14B illustrate another embodiment in which a needle assembly 12, 13 of the tattoo cartridge 10 is coupled to a low frequency oscillator (a rotary disk driven by a motor, with an off-centre pin) 122 and a high frequency oscillator (ultrasonic transducer) 82 of a tattoo machine. In this embodiment, a flexible coupling allows low frequency oscillations generated by the low frequency oscillator 122 to be transferred to the needle assembly 12, 13, effectively “bypassing” the ultrasonic transducer 82. As shown in FIG. 14, the needle bar 13 comprises a flange 125 at its free end that couples to a spring-loaded interface 126 of the ultrasonic transducer 82.

In this embodiment, the coupling further functions to hold the needle assembly 12, 13 coaxially within the shell 11, and thus as can be seen in FIG. 14, the tattoo cartridge does not include a limit device or bush for that purpose. As shown in FIG. 14, in this embodiment, collar 124 is attached to the needle assembly at a nodal point, cap 16 closes the distal end of the shell 11, and elastic membrane 15 is attached between the collar 124 and cap 16.

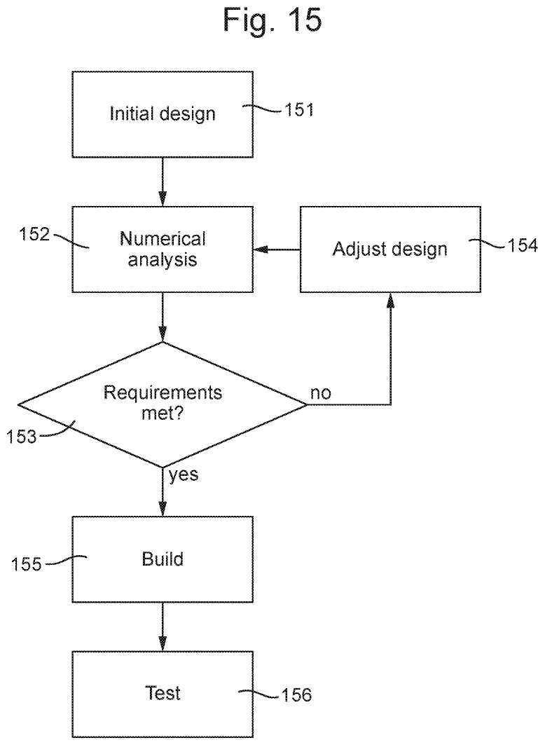

The position and/or extent of a node can be configured with the aid of numerical analysis, and confirmed experimentally. FIG. 15 illustrates a design process according to embodiments. As shown in FIG. 15, an initial needle assembly design may be generated (at step 151), for example based on wavelength theory calculations.

For example, in the case of a transducer having a half-wavelength design (i.e. where anti-nodes occur at the ends of the transducer, and a single node occurs in between the transducer ends), an initial needle assembly design may have a half-wavelength design, e.g. with a length L equal to half a wavelength, L=λ/2. For example, in the case of the transducer operating resonant frequency being 50 kHz, and the needle assembly being made of steel with speed of sound being 4943 m/s, an initial length of a needle assembly having a matching 50 kHz resonant frequency may be determined to be L=(4943/50e3)/2=49.4 mm. This calculation assumes constant needle assembly diameter, but it will be appreciated that an initial design can be adapted to include desired physical features, e.g. as described above.

Other arrangements, such as full wavelength design, are possible.

An initial design may then be modelled numerically, e.g. by finite element analysis (at step 152), and the numerical modelling may be used to determine whether the modelled design performs as desired, e.g. with a node occurring at a desired location. If the numerical modelling indicates that the design operates as desired, a needle assembly/tattoo device may be built according to the design (at step 155), and tested (at step 156), e.g. by electro-mechanical and vibration analysis. If the numerical modelling indicates that a design does not operate as desired, the design may be adjusted (at step 154), and the adjusted design subjected to numerical analysis (at step 152), etc. For example, the position of the nodal plane may be adjusted by adjusting the mass distribution on either side of the nodal plane. A needle assembly and tattoo device can thereby be configured by an iterative design process. However, as will be appreciated by those skilled in the art, other processes are possible.

Although the present invention has been described with reference to preferred embodiments, it will be understood by those skilled in the art that various changes in form and detail may be made without departing from the scope of the invention as set forth in the accompanying claims.

1. A tattoo device comprising:
a tattoo cartridge comprising a needle assembly and a housing assembly; and a tattoo machine configured to oscillate the needle assembly relative to the housing assembly at a first frequency, and to simultaneously vibrate the needle assembly at a second, higher frequency;
wherein the needle assembly is connected to the housing assembly by a connection assembly such that the needle assembly can oscillate with respect to the housing assembly at the first frequency; and
wherein the needle assembly is configured such that in response to being vibrated at the second, higher frequency, an acoustic standing wave can occur along the needle assembly, and wherein the connection assembly is attached to the needle assembly substantially at one or more nodal points or regions of the acoustic standing wave.

2. The tattoo device of claim 1, wherein the connection assembly comprises an elastic member.

3. The tattoo device of claim 2, wherein the tattoo cartridge is configured such that the elastic member is pre-strained when the tattoo cartridge is connected to the tattoo machine such that the elastic member provides a force for acoustically coupling the needle assembly to the tattoo machine.

4. The tattoo device of claim 2, wherein the elastic member is an impermeable membrane.

5. The tattoo device of claim 2, wherein the elastic member is attached to the needle assembly substantially at a nodal point or region.

6. The tattoo device of claim 2, wherein the connection assembly comprises a first member attached to the needle assembly substantially at a nodal point or region, and the elastic member is attached to the first member.

7. The tattoo device of claim 6, wherein the first member is configured to engage an inner surface of the housing assembly to hold the needle assembly coaxially within the housing assembly.

8. The tattoo device of claim 1, wherein the tattoo cartridge comprises one or more second members configured to hold the needle assembly coaxially within the housing assembly.

9. The tattoo device of claim 1, wherein the connection assembly is attached to the needle assembly at a nodal region of the needle assembly that has a greater mass per unit length than other regions of the needle assembly.

10. The tattoo device of claim 1, wherein the first frequency is a frequency in the range 1-1000 Hz, and the second frequency is a frequency in the range 5-200 kHz.

11. The tattoo device of claim 1, wherein:
the needle assembly extends from a first end configured to couple to the tattoo machine, to a second, sharps end; and
the needle assembly comprises metal along substantially its entire length.

12. The tattoo device of claim 11, wherein the metal comprises stainless-steel, titanium, nickel titanium, or aluminium.

13. The tattoo device of claim 1, wherein:
the needle assembly comprises a tattoo needle attached to a needle bar;
an end of one of the tattoo needle and the needle bar comprises a cavity; and
an end of the other of the tattoo needle and the needle bar is received within the cavity.

14. The tattoo device of claim 13, wherein the cavity is formed in the needle bar and is configured to receive different types of tattoo needle.

15. The tattoo device of claim 1, wherein the second, higher frequency is a resonant frequency of an oscillator of the tattoo machine and of the needle assembly.

16. The tattoo device of claim 1, wherein the tattoo machine comprises a flexible coupling, and the needle assembly is coupled to the flexible coupling.

17. The tattoo device of claim 1, wherein the needle assembly comprises a rounded end, and the tattoo machine comprises a complementary socket configured to receive the rounded end to acoustically couple the needle assembly to the tattoo machine.

18. A tattoo cartridge for use with a tattoo machine that comprises one or more oscillators configured to oscillate a needle assembly of the tattoo cartridge at a first frequency, and to simultaneously vibrate the needle assembly at a second, higher frequency; the tattoo cartridge comprising:
a housing assembly; and
a needle assembly connected to the housing assembly by a connection assembly such that the needle assembly can oscillate with respect to the housing assembly at the first frequency;
wherein the needle assembly is configured such that in response to being vibrated at the second frequency, an acoustic standing wave can occur along the needle assembly, and wherein the connection assembly is attached to the needle assembly substantially at one or more nodal points or regions of the acoustic standing wave.

19. A tattoo machine comprising:
a cartridge connector configured to receive a tattoo cartridge comprising a needle assembly and a housing assembly; and
one or more oscillators configured to oscillate a needle assembly of a tattoo cartridge connected to the cartridge connector at a first frequency, and to simultaneously vibrate the needle assembly at a second, higher frequency.

20. A method of operating the tattoo device of claim 1, the method comprising connecting the tattoo cartridge to the tattoo machine.

21. A method of applying a tattoo, the method comprising applying the tattoo using the device of claim 1.

22. A method of manufacturing the tattoo cartridge of claim 18, the method comprising:
attaching the connection assembly to the needle assembly substantially at the one or more nodal points or regions.
